(19) United States
(12) Patent Application Publication
(10) Pub. No.: US 2004/0242477 A1
(43) Pub. Date: Dec. 2, 2004

(54) Protein modification reagents

(76) Inventors: Richard Anthony Godwin Smith, Essex (GB); Jason Richard Betley, Essex (GB)

Correspondence Address: HELLER EHRMAN WHITE & MCAULIFFE LLP, 1666 K STREET,NW, SUITE 300, WASHINGTON, DC, 20006, US

(21) Appl. No.: 10/483,568

(22) PCT Filed: Jul. 15, 2002
(86) PCT No.: PCT/GB02/03210

(30) Foreign Application Priority Data
Jul. 13, 2001 (GB) 0117193.3

(51) Int. Cl. C07K 14/47; A61K 38/17

(52) U.S. Cl. 514/12; 530/408

(57) ABSTRACT

The invention relates to a protein modification reagent capable of introducing aldehyde or ketone functions into proteins. These compounds can be used to modify peptides in a site-specific and pharmaceutically acceptable manner. Also described are methods for modifying peptides and their use in pharmaceutical compositions.

This invention relates to protein modification reagents. More particularly it relates to protein modification reagents capable of introducing aldehyde or ketone functions into proteins in a site-specific and pharmaceutically acceptable manner.

Evidence has recently emerged that the presence of free carbonyl functions on polypeptide chains or glycoprotein carbohydrate can mediate a variety of biologically important processes, particularly ones associated with modulation of the immune response. Several reports have implicated the generation of aldehyde functions through oxidation of carbohydrate and by other mechanisms in the enhancement of adaptive immune responses. (e.g Zheng et al, Science, 256: 1560-3, 1992). Allison & Fearon (Eur J Immunol. 30:2881-7, 2000) showed that attachment of glycolaldehyde to poorly immunogenic antigens rendered them effective immunogens possibly by enhancing the presentation of antigen to T-lymphocytes by macrophages and dendritic cells. Salmi et al (Immunity 14: 265-76, 2001) proposed that the amine oxidase activity associated with the adhesion protein vascular adhesion protein-1 (VAP-1) functions to deaminate primary amine groups on cell surface proteins and to permit reversible Schiff base formation between the resulting cell-bound protein/carbohydrate aldehyde and further primary amines on other cells, thus giving an additional adhesion mechanism possibly significant in the extravasation of lymphocytes.

These observations suggest that the systematic introduction of aldehydes into proteins could be of value in creating more effective vaccines, in stimulating the localisation of lymphocytes at sites of infection, and generally in stabilising biological agents at a defined cellular site. In addition, an aldehyde or ketone functionality in a protein permits novel protein conjugation and derivatisation modalities to be developed.

However, only limited chemical options exist for creating aldehyde or ketone derivatives of proteins and they cannot be controlled precisely, rendering them unsuitable for the development of therapeutic agents. For example, the reaction of glycolaldehyde with protein primary amines results in an Amadori rearrangement of the initial Schiff base and the regeneration of the aldehyde function as an amino-ethanal (aldoamine) derivative. This can then react with further primary amino groups in the protein, resulting in intra- and inter-molecular cross-linking (MacDonald & Pepper, Methods Enzymol. 231: 287-308, 1994). Periodate oxidation of sugars attached to proteins also gives rise to intrinsically unstable di-aldehyde functions and care is required to avoid protein cross-linking (Allison & Fearon, Eur J Immunol. 30:2881-7, 2000).

The present invention therefore provides for chemical reagents which can be introduced into a protein at defined sites in the polypeptide chain under conditions which do not result in a free carbonyl (aldehyde or ketone) group being generated. The modified protein can be isolated, stored, and formulated. Furthermore, the invention provides a modified protein capable of regenerating one or more free aldehyde or ketone groups under physiological conditions.

The compounds of the invention comprise the following elements:

W-[D]-([L]-([X]ₚ)ₘ)ₙ (I)

wherein:

W is a protein attachment group exemplified by but not limited to:

2- or 4-dithiopyridyl, N-maleimido, halo-alkyl or vinyl sulphone groups for reaction with free thiols of cysteine residues in proteins

Cysteine or homocysteine linked through its carboxyl group to D—that is, with free thiol and amino groups capable of undergoing specific coupling to thiolester functions generated within proteins

A thiolester group capable of reacting specifically with N-terminal cysteine in polypeptides.

An N-oxysuccinimido, pentafluorophenyl or other activated ester of a carboxylic acid capable of reaction with free amino groups in proteins.

D is a nodal structure with valency n, capable of linking W to n copies of L. n is preferably 1 to 6, more preferably 1 to 3. Where n is 1, D may incorporated into L. Examples of D include tris-(hydroxymethyl)aminomethane (n=3 with L linked to the hydroxy groups and W to the amino group).

L is a linker or spacer region. L is preferably linear although branched units (equivalent to D with m preferably 2 or 3) may also be employed to a give a dendritic structure with large numbers of X.

Examples of L include but are not limited to oligomethylene units (preferably less than (CH₂)₈), oligopeptides (preferably less than 10 amino acids) and oligo-oxyethylene units (preferably less than 100). L can also be a combination of these units.

It will be clear to those skilled in the art that D and L are a means to link W to X and if a compound is linear and not branched only L is required.

X is a protected precursor of a carbonyl function, preferably an aliphatic aldehyde. The choice of protecting group depends on the conditions under which the carbonyl function is to be regenerated and the rate at which this process needs to occur.

In general, preferred forms of X comprise the acetals —CH(OR)₂ which will be stable under aqueous alkaline conditions (i.e. at pH>8.5) and labile under acid conditions (pH<6.5).

Further X can also be a diol function, which yields an aldehyde after treatment with an oxidant such as NaIO₄.

R is preferably lower alkyl such as methyl or ethyl. The protecting group may optionally be enzyme- or photo-activated. X will normally contain a single aldehyde or ketone derivative (i.e p=1 in (I)) but derivatives of dialdehydes, such as malondialdehyde (p=2), may also be employed.

The invention also provides for protein derivatives containing aldehyde or ketone precursors, particularly derivatives of antigens and immunogens. Such precursors are stable in that they can be formulated for intramuscular, subcutaneous or intradermal administration to humans and animals. Such formulations may be, preferably, lyophilised. When required the formulation may be reconstituted using pharmaceutically acceptable media so that the reconstituted solution is at a pH where the aldehyde/ketone precursor is stable for several hours at ambient temperature.

WO98/02454 describes soluble derivatives of soluble polypeptides, which comprise two or more heterologous membrane binding elements with low membrane affinity covalently associated with the polypeptide, the elements being capable of interacting, independently and with thermodynamic additivity, with components of cellular or artificial membranes exposed to extracellular fluids. That invention thus permits the localization of a therapeutic agent at an outer cellular membrane surface. In a further embodiment, the present invention therefore provides for a soluble derivative of a soluble polypeptide according to WO98/02454 incorporating or linked to a structure such as (I) or elements of (I) containing, minimally, the protected carbonyl function X.

This latter embodiment provides for agents capable of displaying a free carbonyl function on the surface of a cell or lipid bilayer structure. Such derivatives can be used as adjuvants for whole-cell or liposomal vaccines. Further, they may also be applied to delivery of cell- or liposome-based therapeutics or gene therapy agents to the vascular endothelium.

In a further embodiment, the invention provides for methods of linking polypeptides to each other and to non-polypeptide entities using aldehyde- or ketone-based chemistry. Thus, for example, orthogonal coupling of two polypeptides, proteins or protein fragments may be accomplished by reaction of one component with a compound (I) and subsequent reaction of this derivative with another compound or peptide derivatised with an aldehyde-reactive function such as a hydrazine or hydrazone. Conjugates of this type have a wide variety of uses including but not restricted to diagnostic reagents using enzyme-based detection and drug-antibody conjugates such as targeted cytotoxic agents.

The use of the terms “protein” and “peptide” or “polypeptide” are used interchangeably in this specification to refer to a compound that comprises amino acid residues.

### DRAWINGS

FIG. 1: Schematic of the synthesis of APT1404 (4-Amino-[(2-(2-pyridyldithio)aminoethyl) succinamido]butyraldehyde diethyl acetal).

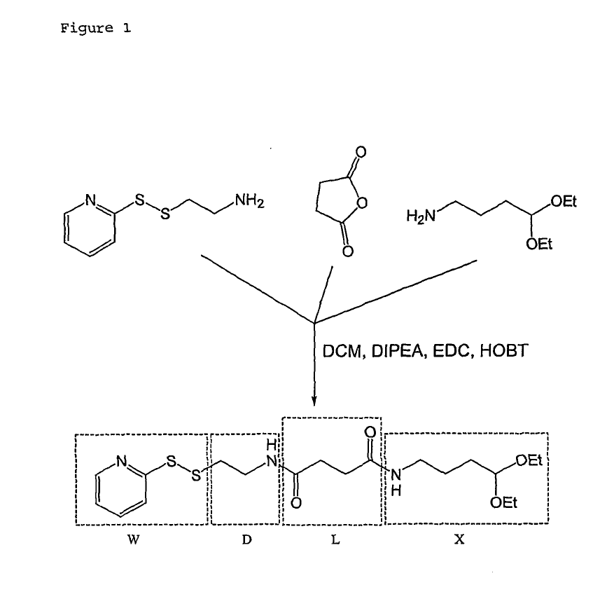

FIG. 2: Schematic of the synthesis of APT2494 (4-Amino-(3-[2-pyridyldithio]propionamido-polyoxyethylene-propionamido)butyraldehyde diethyl acetal).

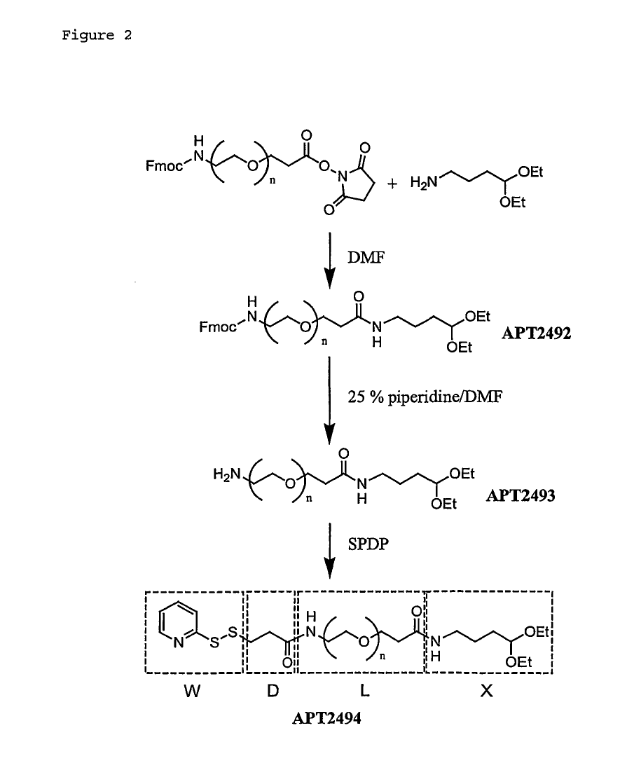

FIG. 3: A schematic of the modification of a peptide (APT154) with a compound of the invention (APT2494) to produce a modified peptide containing an aldehyde/ketone precursor.

FIG. 4: An illustration of examples of the group W where 1. is 2-dithiopyridyl; 2. is 4-dithiopyridyl; 3. is a haloalkyl; 4. is homocysteine; 5. is pentafluorophenyl ester; 6. is vinyl sulfone; 7 is cysteine; 8. is thiolester; and 9. N-oxysuccinamido.

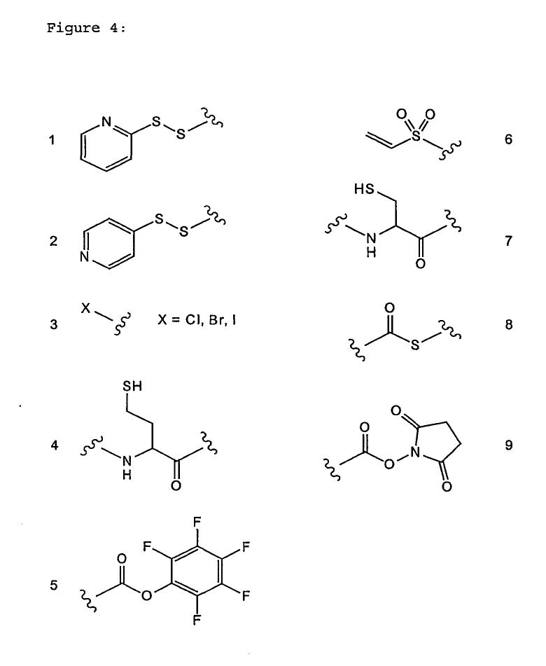

### GENERAL METHODS

These general methods provide background chemistry techniques and are well know to those skilled in the art.

1. Site-Specific Introduction of Thiols Into Recombinant Proteins

A thiol may be introduced into a recombinant protein at any position by manipulation of the DNA encoding the gene such that the amino acid cysteine is inserted at the desired position in place of the native amino acid. Cysteine is the only amino acid which possesses a free thiol function. It is encoded by a UGU or UGC codon on an mRNA molecule.

To introduce a cysteine into a recombinant protein, the cDNA encoding the protein within an expression plasmid can be altered to introduce a cysteine codon, via the process of site-directed mutagenesis.

A number of processes exist to perform site directed mutagenesis. All these rely on the annealing of a short DNA oligonucleotide that contains the necessary bases to effect the change flanked by bases complementary to the parent molecule such that the oligonucleotide forms conventional base pairs. Via in vitro extension reactions and a step to select against parental DNA, the product of the reaction is a DNA molecule identical to the parent with the exception of those bases deliberately altered to introduce a cysteine codon.

2. Reduction of Disulphides and Modification of Thiols in Proteins

In order to introduce an aldehyde or ketone precusor into a protein using thiol-based chemistry, it may be necessary to carry out selective reduction of disulphides bonds in proteins or antigens. During the isolation and purification of multi-thiol proteins, in particular during refolding of fully denatured multi-thiol proteins, inappropriate disulphide pairing can occur. In addition, even if correct disulphide pairing does occur, it is possible that a free cysteine in the protein may become blocked, for example with glutathione or cysteine. These derivatives are generally quite stable. In order to make them more reactive, for example for subsequent conjugation to another functional group, they need to be selectively reduced, with for example dithiothreitol (DTT) or Tris (2-carboxyethyl) phosphine.HCl (TCEP) then optionally modified with a function which is moderately unstable. An example of the latter is Ellman's reagent (DTNB) which gives a mixed disulphide. In the case where treatment with DTNB is omitted, careful attention to experimental design is necessary to ensure that dimerisation of the free thiol-containing protein is minimised. Reference to the term “selectively reduced” above means that reaction conditions eg. duration, temperature, molar ratios of reactants have to be carefully controlled so that reduction of disulphide bridges within the natural architecture of the protein is minimised. The following general examples illustrate the type of conditions that may be used and that are useful for the generation of free thiols and their optional modification. The specific reaction conditions to achieve optimal thiol reduction and/or modification are ideally determined for each protein batch and such determination is within the normal skill of those in the art.

TCEP may be prepared as a 20 mM solution in 50 mM Hepes (approx. pH 4.5) and may be stored at −40° C. DTT may be prepared at 10 mM in sodium phosphate pH 7.0 and may be stored at −40° C. DTNB may be prepared at 10 mM in sodium phosphate pH 7.0 and may be stored at −40° C. All of the above reagents are typically used at molar equivalence or molar excess, the precise concentrations ideally identified experimentally. The duration and the temperature of the reaction are similarly determined experimentally. Generally the duration would be in the range 1 to 24 hours and the temperature would be in the range 2 to 30° C. Excess reagent may be conveniently removed by buffer exchange, for example using Sephadex G25. A suitable buffer is, for example, 0.1M sodium phosphate (pH 7.0).

3. Non Site-Specific Introduction of Thiols Into Proteins

If no specific cysteine is engineered into the protein (vide supra) then it is possible to introduce one or more of them in a number of ways. Surface lysine residues can be reacted with 2-iminothiolane (Traut's Reagent) to generate free thiols. The extent of this reaction can be controlled by molar equivalency, time, and the nature of the buffer to introduce more or fewer free thiols as required. The number of free thiols introduced may be easily quantified by the Ellman-type assay, whereby free thiols reduce a chromogenic disulfide. Other methods to introduce thiols include reaction of the protein with SPDP (3-(2-pyridyldithio)propionic acid N-oxysuccinimide ester), a heterobifunctional molecule, one end of which reacts with amino groups in surface lysines of proteins. The pendant 2-pyridyl disulfide, thus installed, is quantifiable by treatment of the preparation with TCEP and assay of the 2-thiopyridine formed. This compound has a known extinction coefficient at 343 nm.

Another reagent of this type is SATA (S-acetyl thioglycollic acid N-oxysuccinimide ester) which introduces a protected thioacetyl group. This can be hydrolysed under mild conditions to give a free thiol.

Other methods exist to achieve non-specific thiol introduction and are well known to those skilled in the art.

The following examples illustrate the invention.

### EXAMPLE 1

### Synthesis of APT1404 (4-Amino-[(2-(2-pyridyldithio)aminoethyl)succinamido]butyraldehyde Diethyl Acetal)

2-Pyridyl-dithioethylamine dihydrochloride (1.00 g) and succinic anhydride (386 mg) were suspended in dichloromethane (15 mL). Diisopropylethylamine (1 mL) was added and the mixture stirred over 1 h. 4-Aminobutyraldehyde diethyl acetal (667 μL), 1-[3-(dimethylamino)propyl]-3-ethylcarbodiimide hydrochloride (1.11 g), and 1-hydroxybenzotriazole (100 mg) were added and the mixture stirred over a further 2 h. The reaction mixture was extracted with saturated sodium bicarbonate and brine, dried over magnesium sulfate, and evaporated to yield a colourless oil. Silica gel chromatography using 0.2% triethylamine in dichloromethane with a methanol gradient afforded APT1404 as a stiff glass. ¹H NMR (CDCl₃) 1.20 (m, 6H), 1.50-1.70 (m, 4H), 2.45-2.65 (m, 4H), 2.90 (t, 2H), 3.15-3.25 (m, 2H), 3.40-3.70 (m, 6H), 4.45 (dt, 1H), 6.25 (br, 2H), 7.10-7.20 (m, 1H), 7.45-7.55 (m, 1H), 7.55-7.65 (m, 1H), 8.50-8.55 (m, 1H).

### EXAMPLE 2

### Synthesis of APT2494 (4-Amino-(3-[2-pyridyldithio]propionamido-polyoxyethylene-propionamido)butyraldehyde Diethyl Acetal)

9-Fluoroenylmethoxycarbonyl-NH-polyethyleneglycol(3400)—N-succinimidyl propionate (34 mg, 10 μmol) was dissolved in dry dimethylformamide (600 μL). 4-aminobutyraldehyde diethyl acetal (2.13 μL, 11 μmol) was added and the mixture stirred for 15 minutes. The product (APT2492) was purified by HPLC using a gradient of 10-90% buffer B in buffer A over 20 minutes and a Jupiter C18, 250×10 mm, 300 Å column running at a flow rate of 5 mL/min (buffer A: 0.1% trifluoroacetic acid; buffer B: 90% acetonitrile, 0.1% trifluoroacetic acid). The volatile components were evaporated under reduced pressure and the aqueous solution lyophilised. Retention time 15.9 min; MALDI-TOF mass spectrometry showed a group of peaks centred around 3750 (starting material Fmoc-NH-PEG3400-CO₂—NHS MALDI-TOF mass spectrometry showed a group of peaks centred around 3700).

APT2492 (20 mg, 6 μmol) was dissolved in 600 μL of a solution of 25% piperidine in dimethylformamide. The mixture was stirred for two hours and the product (APT2493) isolated and purified as for APT2492. Retention time 12.2 min; MALDI-TOF mass spectrometry showed a group of peaks centred around 3550.

APT2493 (10 mg, 3 μmol) was dissolved in 300 μL of 50 μM phosphate buffer, pH 7. To this was added 200 μL of a 0.024 M solution of N-succinimidyl 3-[2-pyridyldithio]propionate in dimethylsulfoxide (4.8 μmol). The mixture was stirred for two hours and the product isolated and purified as for APT2492. Retention time 11.7 min; MALDI-TOF mass spectrometry showed a group of peaks centered around 3750.

### EXAMPLE 3

### Linking of APT2494 to a Thiol-Containing Protein

APT154, a 22 kDa protein containing a single unpaired thiol at the C-terminus (50 μL of a 100 μM solution in PBS, 5.0 nmol) was treated overnight at 20° C. with a solution of tris-2-carboxyethyl phosphine (TCEP) (3 molar equivalents of a 10 mM solution in PBS). APT2494 (20 molar equivalents of a 5 mM solution in PBS) was added and the mixture incubated at 20° C. over 3 h. Conversion to derivatised product was evidenced by gel shift on a polyacrylamide gel, by MALDI mass spec, and by size exclusion chromatography. Purification was achieved by one of a number of possible methods using conditions of pH>7.

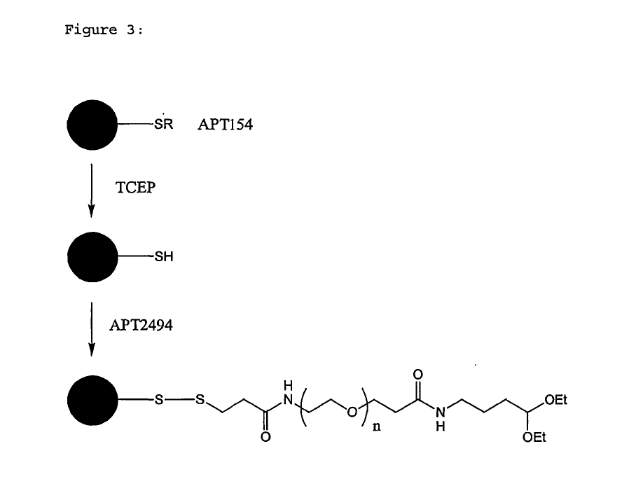

This process is illustrated in FIG. 3.

1. A compound of the formula:
W-[D]-([L]-([X]ₚ)ₘ)ₙ (I)
wherein:
W is a protein attachment group having a reactive thiol group;
D is a nodal structure with valency n and is optional;
n is 1 to 6;
p is 1 to 3;
m is 1 to 4;
L is a linker or spacer region; and
X is a protected precursor of a carbonyl function, preferably an aliphatic aldehyde.

2. The compound according to claim 1, wherein W is
2- or 4-dithiopyridyl, N-maleimido, halo-alkyl or vinyl sulphone groups, cysteine or homocysteine, a thiolester, an N-oxysuccinimido, pentafluorophenyl or other activated ester of a carboxylic acid.

3. The compound according to claim 1, wherein X is an acetal such as the group CH(OR)₂, and R is a lower alkyl such as methyl or ethyl.

4. The compound according to claim 1, wherein X is a diol function.

5. The compound according to claim 1, wherein L is linear or branched.

6. The compound according to claim 1, wherein L is selected from the group consisting of: oligomethylene units, oligopeptides, and oligo-oxyethylene units.

7. The compound according to claim 1, wherein the compound is branched and D is tris-(hydroxymethyl) aminomethane.

8. The compound according to claim 1, wherein the compound is 4-Amino-[(2-(2-pyridyidithio)aminoethyl) succinamido]butyraldehyde diethyl acetal or 4-Amino-(3-[2-pyridyldithio]propionamido-polyoxyethylene-propionamido)butyraldehyde diethyl acetal.

9. The compound according to claim 1 for modifying a peptide, wherein the peptide is modified to include an aldehyde or ketone precursor.

10. A process for modifying a peptide, wherein the peptide is modified to include an aldehyde or ketone precursor using a compound according to claim 1 comprising the steps of:
i. introducing a free thiol into the peptide;
ii. admixing the peptide with a compound having a reactive thiol according to claim 1;
iii. reacting the free thiol group with the reactive thiol group; and
iv. separating the modified peptide from a reaction mixture.

11. A peptide obtainable by the process according to claim 10.

12. A peptide obtained by the process according to claim 10, wherein the peptide is an antigen or immunogen.

13. The process according to claim 10, wherein the modified peptide is formed by spontaneous air oxidation between the free thiol group of the peptide and another free thiol group present in W.

14. A peptide modified by the process according to claim 10, wherein the peptide contains an aldehyde or ketone precursor.

15. The peptide according to claim 14, wherein the modified peptide is capable of regenerating one or more free aldehyde or ketone groups under physiological conditions.

16. A pharmaceutical composition comprising a modified peptide made by a method according to claim 10 and a pharmaceutically acceptable excipient.

17. A process for conjugating a peptide moiety to another peptide or non-peptide moiety using aldehyde- or ketone-based chemistry, wherein coupling of the two moieties is accomplished by reaction of one moiety with a compound (I) and the reaction of this derivative with the other moiety derivatised with an aldehyde-reactive function or a ketone-reactive function.

18. A process for conjugating a peptide moiety to another peptide or non-peptide moiety, wherein the two moieties are modified by a compound according to claim 1, and the modified moieties are admixed and allowed to react, thereby forming a conjugated peptide moiety.

19. A modified peptide, wherein the peptide is modified by a compound according to claim 1, wherein the peptide is an antigen or immunogen.
